(19) United States
(12) Patent Application Publication
(10) Pub. No.: US 2023/0403989 A1
(43) Pub. Date: Dec. 21, 2023

(54) PLANTER SYSTEM ENHANCING GROWTH, CARE AND MAINTENANCE OF PLANTS

(71) Applicant: KEVIN FITZGERALD JOHNSON, SPENCER, VA (US)

(72) Inventor: KEVIN FITZGERALD JOHNSON, SPENCER, VA (US)

(21) Appl. No.: 18/212,539

(22) Filed: Jun. 21, 2023

Related U.S. Application Data
(60) Provisional application No. 63/354,140, filed on Jun. 21, 2022.

(51) Int. Cl. A01G 9/02; A01G 29/00; A01G 27/00; A01G 27/06; A01K 67/033

(52) U.S. Cl. CPC A01G 9/02; A01G 29/00; A01G 27/008; A01G 2009/003; A01K 67/033; A01G 9/028; A01G 27/06

(57) ABSTRACT

A planter system including a base container and a transparent water evacuation tube located at a bottom portion of the base container. The transparent water evacuation tube is movable from a position pointing up holding water in the base container and a position pointing down to drain water from the base container. A platform/transplanter insert is located in the base container and has handles that extend upward. The handles are shaped to provide a passage to allow for feeding, watering and aeration.

### CROSS-REFERENCE TO RELATED APPLICATION

This application claims the benefit of U.S. Provisional Patent Application No. 63/354,140, filed on Jun. 21, 2022.

### BACKGROUND

### 1. Field

The present disclosure relates to planter systems, and particularly to planter systems with watering and aeriation.

### 2. Description of the Related Art

Discerning accurate information about a potted plant's health status and environmental conditions is challenging. It is essential to have accurate information so that the container plants can survive and thrive. The plants exist in a restricted, artificial environment where they typically attempt to endure in less than ideal circumstances because of improper and inefficient watering, inefficient and inconsistent feeding, and insufficient aeration. Most container plant enthusiasts, unfortunately, do not have the knowledge, time, patience or understanding of how to create an ideal growing environment for container plants that will allow the plants to thrive. The container plants are not able to develop naturally or to independently create their ideal growing conditions because of the limited environment and improper care.

The plants must also endure traumatic processes when transplanting becomes necessary. Container plants are often traumatized by the rough nature of the universally accepted transplanting process that typically shocks, damages, or kills the transplanted plant.

Container plants, from aesthetic and efficiency standpoints, are routinely and traditionally displayed in bland container/saucer pairings that are neither visually appealing nor efficient from a survival standpoint. The presentation of container plants is very limited due to the relatively few options available.

Present container plant systems do not allow a user to easily acquire accurate information about a plant's health status (root-bound, proper root system, etc.) and/or environmental conditions (hydration, aeration, etc.) or easily adjust as necessary. Thus, a planter system with watering and aeriation solving the aforementioned problems is desired.

### SUMMARY

There is a need for an innovative planter system that assists in creating ideal independent growing environments for container plants that allows plants to survive. It is further beneficial for the planter system to provide a mechanism for plants to be easily moved to different pots with unlimited design options to change the presentation.

A planter system, in one embodiment, includes a base container and a water evacuation tube located at a bottom portion of the base container. The water evacuation tube is movable from a position pointing up holding water in the base container and a position pointing down to drain water from the base container. A platform/transplanter insert is located in the base container and has a hole located at a bottom portion of the platform/transplanter insert. A feeding/watering/aeration tube extends through the platform/transplanter insert and through the hole into the base container.

The water evacuation tube, in some embodiments, is transparent.

The base container, in other embodiments, is transparent allowing for accurate viewing and monitoring of roots.

The planter system can further include an outer container having a slot that accommodates the transparent water evacuation tube when the base container is located in the outer container; and an outer container gate that slides into the slot down to the transparent water evacuation tube to form a completely enclosed outer container perimeter.

The platform/transplanter insert can also include legs extending downward from the bottom portion of the insert leaving space between the bottom portion of the insert and the base container.

The platform/transplanter insert, in other embodiments, includes one or more wicking tubes extending into the base container creating a conduit for water to flow from the base container into the platform/transplanter insert.

The platform/transplanter insert can further include handles for lifting the insert. A plant is easily transplanted using the handles.

The platform/transplanter insert can also include two or more vermiculture containers each having matching holes allowing worms to move back and forth.

The platform/transplanter insert can additionally include two or more mini containers each having holes on a bottom portion allowing water to enter and drain

The planter system can further include a dome covering the platform/transplanter insert to control humidity and aid in plant germination.

A planter system, in an alternate embodiment, includes: a base container; a water evacuation tube located at a bottom portion of the base container, the water evacuation tube movable from a position pointing up holding water in the base container and a position pointing down to drain water from the base container; and a platform insert located in the base container providing a space between the bottom of the base container and the platform insert.

A grower cage can be attached to the platform insert.

The planter insert can further include multiple slots.

One or more grower pots can be located in one or more of the multiple slots.

One or more grower containers can be located in one or more of the multiple slots.

One or more breakaway containers can be located in one or more of the multiple slots.

The planter system can further include a dome covering the platform insert to control humidity and aid in plant germination.

These and other features of the present subject matter will become readily apparent upon further review of the following specification.

### BRIEF DESCRIPTION OF THE DRAWINGS

FIG. 1 is an illustration of a planter system.

FIGS. 2A, 2B and 2C are illustrations of the outer container having a slot and a gate that slides into the slot.

FIG. 2D is an illustration of the outer container having an opening for a water evacuation tube.

FIG. 3 is an illustration of the base container with a water evacuation tube.

FIG. 4 is an illustration of the platform/transplanter insert.

FIG. 5 is an illustration of the platform/transplanter insert with handles.

FIGS. 6A and 6B are illustrations of the vermiculture containers with the base container.

FIGS. 6C and 6D are a top view of multiple mini containers.

FIG. 6E is a front view of multiple mini containers.

FIG. 7 is an illustration of the base container with a dome.

FIG. 8A is an illustration of a platform having a rotating spout.

FIG. 8B is an illustration of a platform insert.

FIG. 8C is a side/front view of the platform insert.

FIGS. 9A and 9B illustrate a platform/cage combo.

FIG. 10A is an illustration greenhouse using a dome.

FIG. 10B is an illustration of a single grower pot placed on an insert in a base container and having a dome.

FIG. 10C is an illustration of multiple individual grower pots each having a dome.

FIG. 11A is an illustration of an individual grower container.

FIG. 11B is an illustration of a grower container filled with soil and a plant.

FIG. 11C is an illustration of an insert with openings for holding multiple grower containers.

FIG. 11D is an illustration of the insert with multiple grower containers placed inside the base container and a dome covering the base container.

FIG. 12A is an illustration of a slotted insert with multiple openings that hold open ended breakaway containers.

FIG. 12B is an illustration of the slotted insert placed inside a base container filled with water.

FIG. 13A is an illustration of a slotted insert with multiple openings that hold immature plants.

FIG. 13B is an illustration of the slotted insert placed inside a base container filled with water.

Similar reference characters denote corresponding features consistently throughout the attached drawings.

### DETAILED DESCRIPTION OF THE PREFERRED EMBODIMENTS

There is a need for a planter system that enhances growth, irrigation, water conservation, feeding, aeriation, root inspection, presentation and transplanting. The planter system should create ideal independent growing environments for container plants that allows plants to survive and thrive. It is further beneficial for the planter system to provide a mechanism for plants to be easily moved to different pots with unlimited design options to change the presentation.

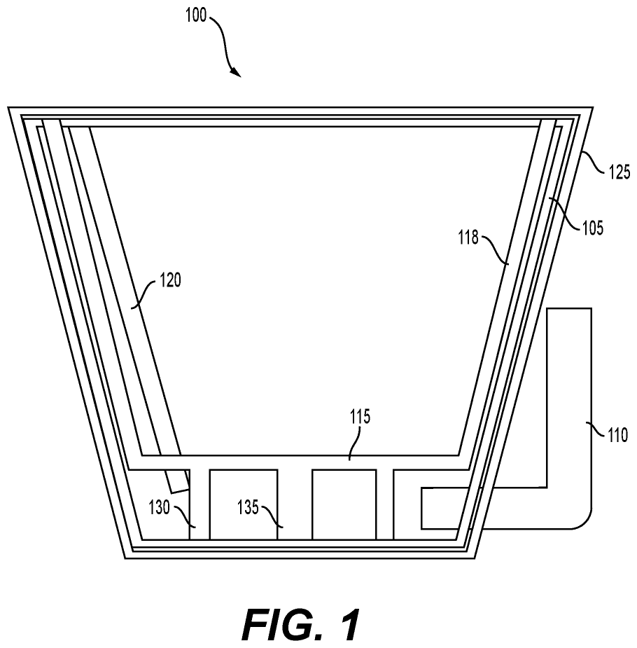

FIG. 1 is an illustration of a planter system 100 having watering and aeration mechanisms. A base container 105 includes a transparent water evacuation tube 110 located at a bottom portion of the base container. The transparent water evacuation tube 110 is movable from a position pointing up holding water in the base container 105, and a position pointing down to drain water from the base container 105. Intermediate positions are also possible. It serves as a water level gauge, water level set, and water inlet/outlet. The base container 105 also includes a through hole to accommodate the transparent water evacuation tube 110. The through hole, in some embodiments, is an elongated cylinder that adds stability and strength.

A platform/transplanter insert 115 is located in the base container 105. A feeding/watering/aeration tube 120 extends through the platform/transplanter insert 115 and through a hole at the bottom of the platform/transplanter insert 115 into the base container 105. The platform/transplanter insert 115 has handles 118 that allows the platform/transplanter insert 115 to be lifted making it easier to transplant a plant.

The base container 105 is transparent, in some embodiments, allowing for accurate viewing and monitoring of roots.

The planter system can further include an outer container 125 having a slot that accommodates the transparent water evacuation tube 110 when the base container 105 is located in the outer container 125. There is also an outer container gate that slides into the slot down to the transparent water evacuation tube 110 to form a completely enclosed outer container perimeter.

The platform/transplanter insert 115 can also include legs 130 extending downward from the bottom portion of the insert leaving space between the bottom portion of the platform/transplanter insert 115 and the base container 105.

The platform/transplanter insert 115 can also include one or more wicking tubes 135 extending into the base container 105 creating a conduit for water to flow from the base container 105 up through the platform/transplanter insert 115.

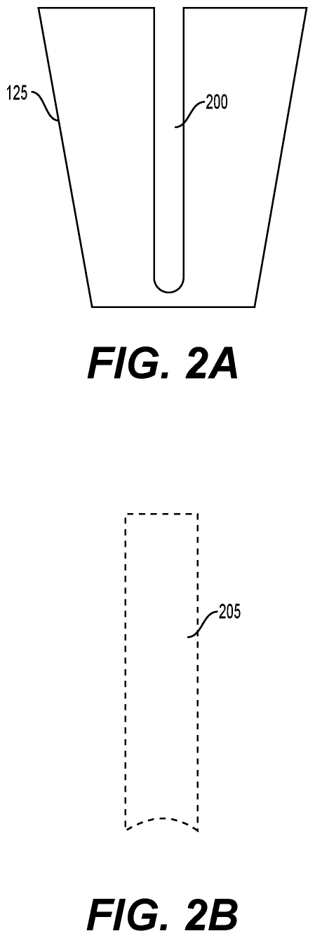

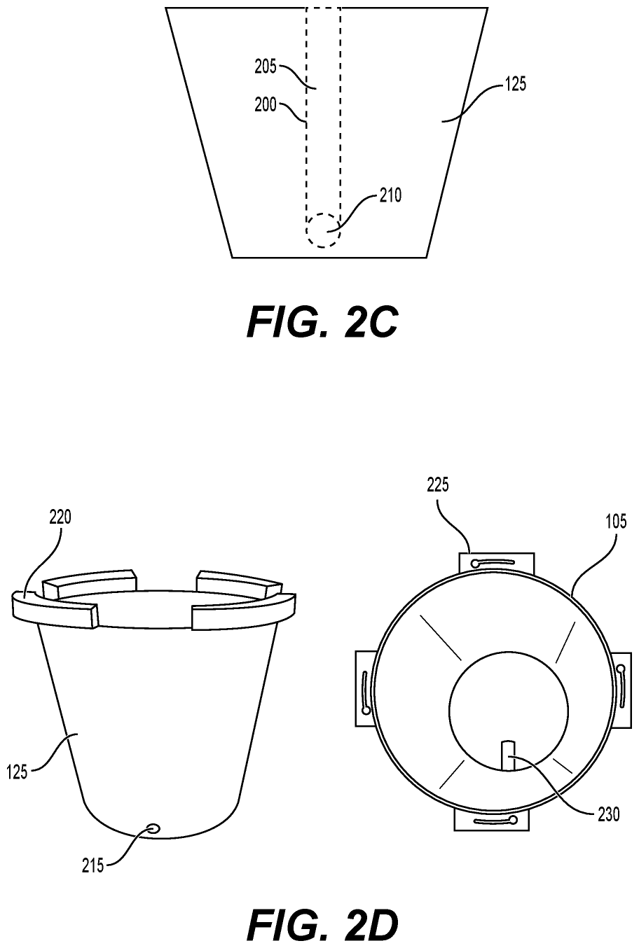

FIG. 2A is an illustration of the outer container 125 having a slot 200 that can accommodate the transparent water evacuation tube 110.

FIG. 2B is an illustration of a gate 205 that fits into the slot 200. The bottom part of the gate 205 allows the the transparent water evacuation tube 110 to protrude from the outer container 125. The gate 205 slides into the slot 200 down to the transparent water evacuation tube 110 to form a completely enclosed outer container perimeter.

FIG. 2C is an illustration of the outer container 125 with the gate 205 positioned in the slot 200. A space 210 is provided for the transparent water evacuation tube 110 when the gate 205 is positioned within the slot 200. A completely enclosed water outer container perimeter is formed when the gate 205 is in the slot 200. Interlocking handles are included on both the base container 105 and the outer container 125. Both have component connection points. The base container 105 will nest inside the outer container 125. Also, the outer container 125 can have handles that seamlessly interlock with the base container. A rotating gauge can be inserted through the outer container into the base container.

FIG. 2D is an illustration of the outer container 125 having an opening 215. The transparent water evacuation tube 110 slides into the opening 215 negating the need for slot 200 and gate 205, as illustrated in FIGS. 2A to 2C. Outer container handles 220 interlock with base container handles 225. This allows for easy alignment of the opening 215 of the outer container 125 and elongated hole tunnel 230 of base container 105. The outer container 125 of FIG. 2D may also be used as a nursery pot by removing the water evacuation tube 110.

From an aesthetic standpoint, the outer container 125 of the planter system 100 can be made of any material and have any design or color scheme. The potential design, material, and color schemes and/or themes are truly unlimited. The containers are also interchangeable which allows a plant to be presented in a new or different container easily and without any trauma.

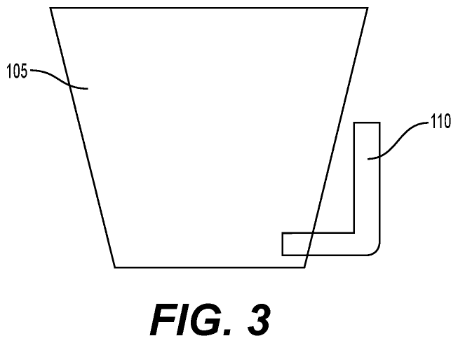

FIG. 3 is an illustration of the base container 105 with a transparent water evacuation tube 110 that rotates from a position pointing up (as illustrated) to a position pointing down. Water is held within the base container 105 when the transparent water evacuation tube 110 is in the position pointing up. Rotating the transparent water evacuation tube 110 to point downward will allow water to be drained from the base container 105. The base container 105 fits within the outer container 125 so that the transparent water evacuation tube 110 slides into the slot 200. The gate 205 slides into the slot 200 down to the transparent water evacuation tube 110 to form a completely enclosed outer container perimeter. The transparent water evacuation tube 110 can be made of two pieces. A first straight tubular piece has a first end that connects/slides horizontally into the base container 105, and a second raised connector end that extends outside of the base container 105. The second vertical piece is a straight tubular piece that connects vertically onto the first straight tubular piece via the second raised connector end. After connected, the requisite right angle is complete. The second vertical piece can vary in length dependent upon the intended use of the container.

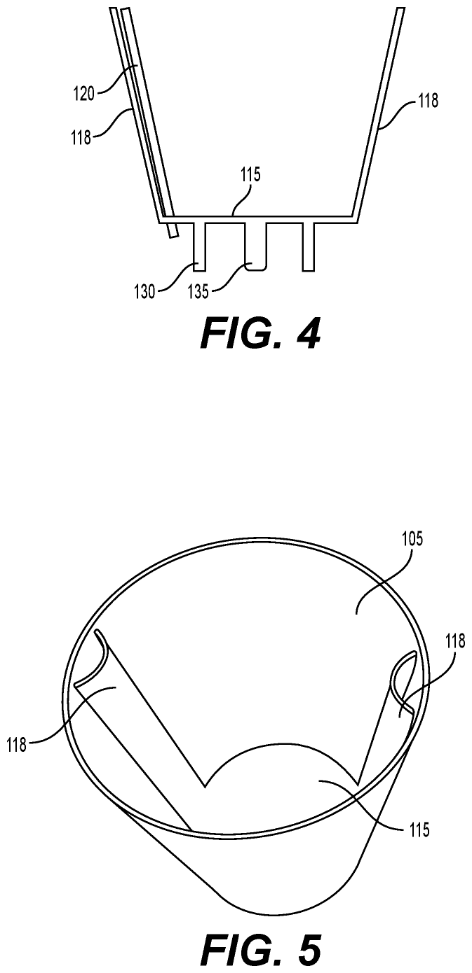

FIG. 4 is an illustration of the platform/transplanter insert 115 having legs 130 extending downward from the bottom portion of the platform/transplanter insert 115 leaving space between the bottom portion of the platform/transplanter insert 115 and the base container 105.

The platform/transplanter insert 115 also has one or more wicking tubes 135 extending into the base container 105 creating a conduit for water to flow from the base container 105 into the platform/transplanter insert 115. Multiple wicking tubes 135 can serve as legs, in some embodiments, making it unnecessary to include legs 130.

Handles 118 allow the platform/transplanter insert 115 to be lifted making it easier to transplant a plant. The platform/transplanter insert 115 allows the plant to sit on a flat surface after being removed from the base container 105. The handles 118 of the platform/transplanter insert 115 are designed to create a channel to let water and/or air flow to the bottom of the base container 105. The handles 118 are also designed to funnel water into the channel when watering volume threatens to overflow the top area when pouring too quickly directly on the plant. After lifting and removing the platform/transplanter insert 115 from the base container 105, the platform handles 118 fold out of the way. The platform/transplanter can have periodic openings around its perimeter that allows water to slowly seep into the lower regions of the base container 105. Base container 105 has handles 118 and component connection points. The handles 118 can be three sided, having two outer segments and a middle segment. The middle segment has the ability to bend while the two outer segments are shorter and do not fully extend to the top or bottom of the base container 105. The design of handles 118 can negate the need for feeding/watering/aeration tube 120.

The innovative planter system 100 as described assists in creating ideal independent growing environments for container plants that allow plants to survive and thrive. Additionally, plants can be easily moved to different pots with unlimited design options to change the presentation.

When watering, the transparent water evacuation tube 110 located on the outside of the container allows the user to monitor the water level as a plant is being watered or as the plant absorbs the water. A user can visually see the water level rise or fall in real time depending on what action is taking place. The tube has the ability to rotate which allows user to remove any excess water if plant is accidentally overwatered or intentionally saturated. Also, water efficiency and conservation are increased because water can be extracted from the reservoir via the rotating tube and used to re-water the plant if necessary or desired. Also, the rotating tube prevents spills when overwatering. Typical plant container saucers fill then overflow immediately after capacity is reached. Additionally, the innovative planter system 100 conserves water because less evaporation takes place than in traditional planters. The water reservoir is exposed to air only via the evacuation tube and the feeding/direct watering/aeration tube 120. The configuration reduces the open surface for water to evaporate. The sum of the area of the two access points is significantly less than the sum total of the area from the circumference/perimeter of typical plant container saucers. Water often flows through the soil into the container saucer. Often the water evaporates into the atmosphere without the plant absorbing the water. In addition, most users will not water their plants when there is water stored in the saucer which often causes the soil to become too dry while waiting for the water to evaporate from the saucer.

The planter system, as disclosed, differs from what currently exists. A better and more effective/efficient container growing environment is created because of better access to real time and precise information regarding watering and feeding and better aeration because both the evacuation tube and the feeding/direct watering/aeration tube allow fresh, circulating air into the root area of the plant. The system allows air to mix with the water before the water is absorbed by the plant via the wicking tube(s)/transplant stand.

When transplanting, user simply grasps the handles and lifts, then removes the plant from the base container. This process is less traumatic for the plant, easier for the user, and less damaging to the container.

FIG. 5 is an illustration of the platform/transplanter insert 115 with handles 118 located within the base container 105. The handles 118, in this embodiment, are modified to have a curved, multi sided shape that creates a space between the handle and sidewall of the base container 105, and allows water and air to enter the bottom of the base container 105. The feeding/watering/aeration tube 120 is not necessary in this embodiment, but can be included.

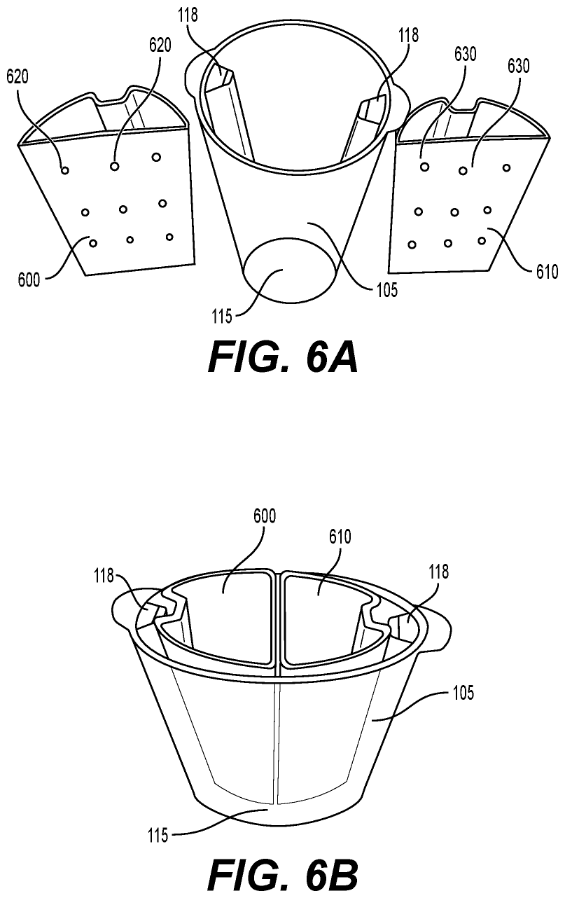

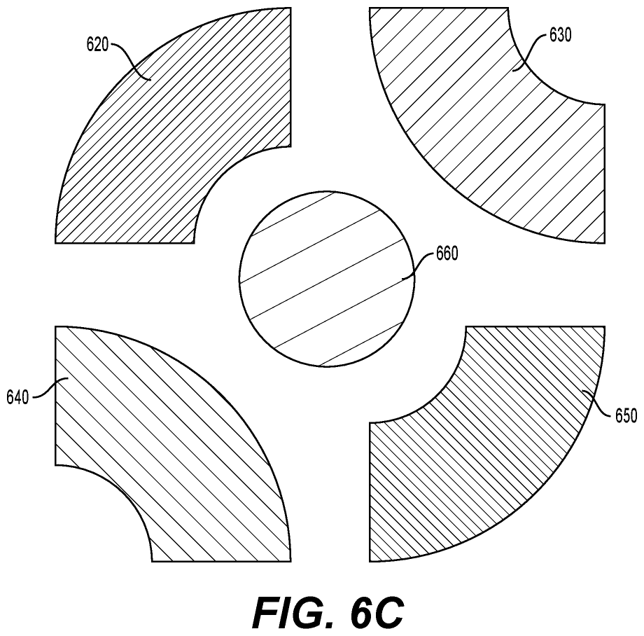

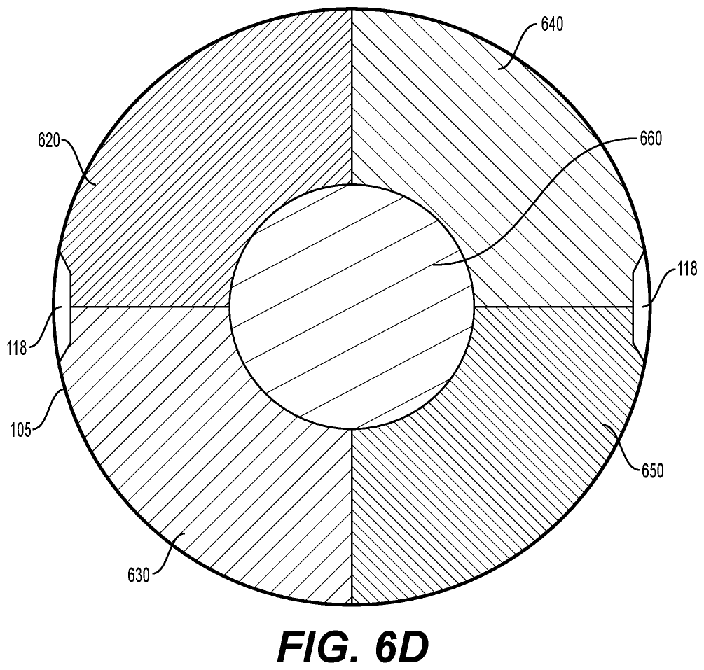

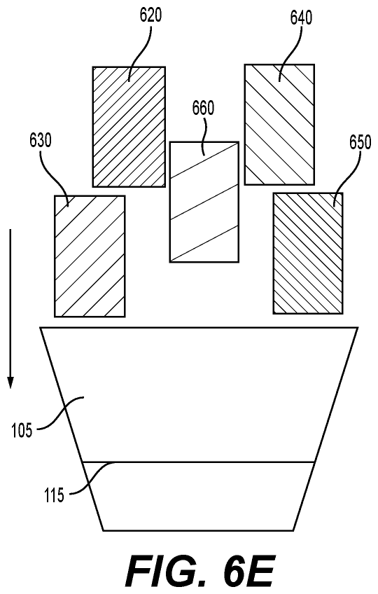

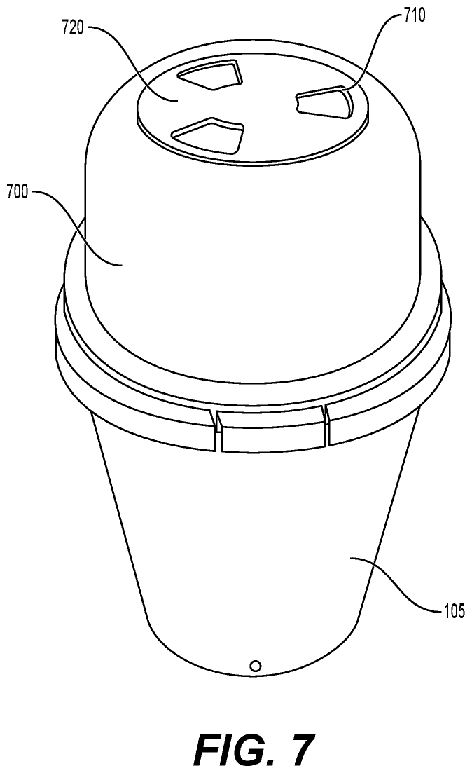

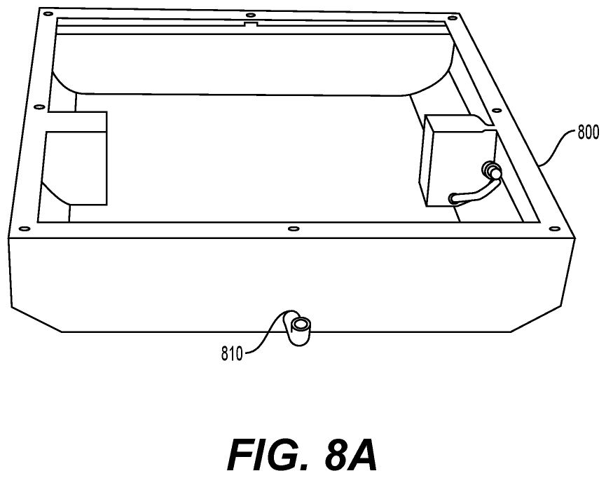

The platform/transplanter insert 115 can also include two or more vermiculture containers (600,610), as illustrated in FIG. 6A, each having matching holes (620,630) allowing worms to move back and forth. The vermiculture containers (600,610) fit within the base container 105 on top of platform/transporter insert 115 with the handles 118 pushed to to sides against the base container 105. The vermiculture containers (600,610) encourage horizontal migration and easier castings extraction. A dome 700, as illustrated in FIG. 7, can be connected to the vermiculture containers (600,610), and can help contain the worms and allow easy access for watering and feeding.

FIG. 6B is an illustration of the vermiculture containers (600,610) located within the base container 105 on the platform/transplanter insert 115. Handles 118 are illustrated as protruding from the base container 105 and can be used to easily transplant a plant from the base container 105.

FIGS. 6C and 6D are a top view of multiple mini containers (620, 630, 640, 650, 660) and FIG. 6E is a front view. Each of the mini containers (620, 630, 640, 650, 660) vertically slides in and out individually and fits into the base container 105. They all rest on the platform/transplanter 115 and fit around the platform handles 118. The tops of each of the mini containers (620, 630, 640, 650, 660) are open but the sides are solid. The bottoms have numerous small openings to allow water to both enter and to drain from the container. The mini containers (620, 630, 640, 650, 660) can be used to host a variety of plants like dish gardens or used as a plant nursery. This configuration is one example and many variations are possible. For example, matching holes can be included in the sides of the mini containers (620, 630, 640, 650, 660) allowing worms to move back and forth; the mini containers (620, 630, 640, 650, 660) can be transparent to allow visual inspection of the roots; or the mini containers (620, 630, 640, 650, 660) can be of different sizes and shapes than those illustrated.

The base container 105, as illustrated in FIG. 7, can further include a dome 700 covering the platform/transplanter insert 115 (not shown) to control humidity and aid in plant germination. The dome 700 attaches to the base container 105 and includes adjustable access ports 710. Seed germination, watering access, feeding access, and worm containment will be improved by including dome 700. A rotating piece 720 that connects to the top of dome 700 can vary the size of the openings in the dome 700. The dome 700 connects to the container via the connection points. It can be made of clear/transparent materials.

The base container 105 fits snugly inside the outer container 125. A platform/transplanter insert 115 with legs 130 and one or more wicking tubes 135 fits snugly inside of the base container 105 (space around sides let water flow through soil into bottom into the reservoir). A transparent rotating water evacuation tube 110 is attached through a hole in the side near the bottom of the base container 105. The platform/transplanter 115 has a feeding/watering/aeration tube 120 attached to a hole in the platform/transplanter insert 115.

The base container 105, transparent rotating water evacuation tube 110, platform/transplanter insert 115, and feeding/watering/aeration tube 120 all work in conjunction to make the unit function properly. Additionally, rollers could be attached to the outer container 125 to make larger units easier to move.

The unit can function without the outer container 125. Users can visually assess the overall health of the plant by viewing the roots growing in the soil in the base container 105 if the base container 105 is transparent. The base container 105 can be decorated/adorned with stickers, slogans, images, etc. in order to personalize the appearance. FIG. 5 shows handles 118 on the platform/transplanter insert 105 as being hollow making the feeding/watering/aeration tube 120 unnecessary.

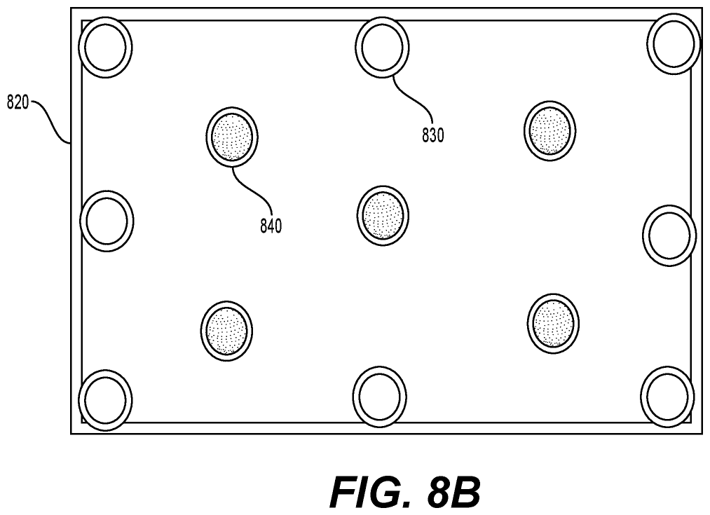

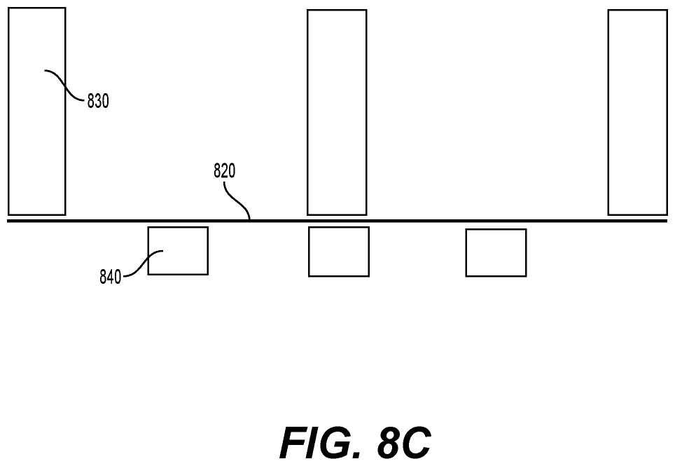

The unit, in an alternate embodiment, can be made large enough to accommodate landscape plants. FIG. 8A is an illustration of a platform 800 having a rotating spout 810. FIG. 8B is an illustration of a platform insert 820 that includes hollow watering tubes 830 and wicking channels/platform supports 840. FIG. 8C is a top view of platform insert 820. The platform insert 820 is placed in the platform 800. The platform 800 can accommodate different inserts, other than platform insert 820, that allow the functionality to change based on what components are used. For example, vermiculture containers (600,610), as illustrated in FIG. 6A, or multiple mini containers (620, 630, 640, 650, 660), as illustrated in FIGS. 6C to 6E, could be implemented as a type of insert for platform 800. The variations are endless.

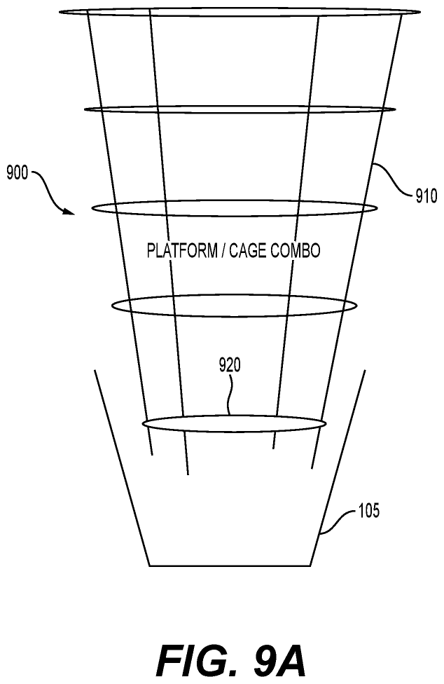

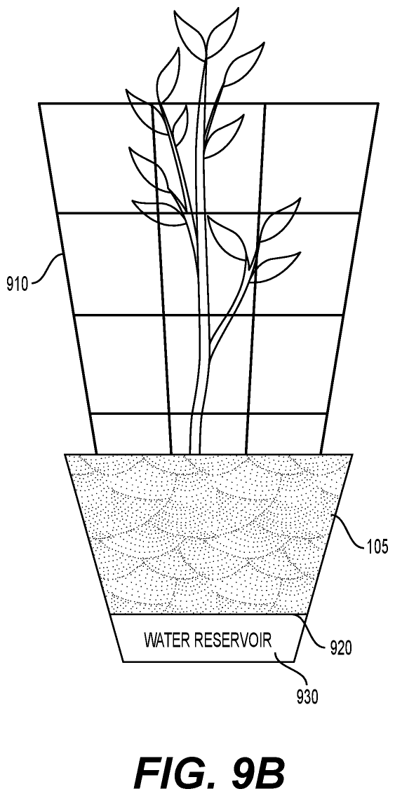

FIGS. 9A and 9B illustrate a platform/cage combo 900 that fits within the base container 105 that includes a rotatable water spout (not illustrated). A grower cage is 910 is attached to a platform 920 forming the platform/cage combo 900. A water reservoir 930 is formed below the platform 920, and the grower cage 910 allows a plant to mature with support. The plant can be removed from the base container 105 by lifting the platform/cage combo 900. The platform/cage combo can be attached to the base container 105.

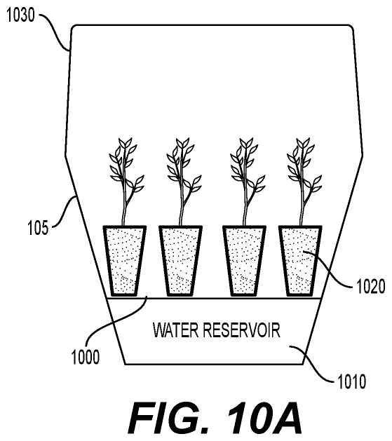

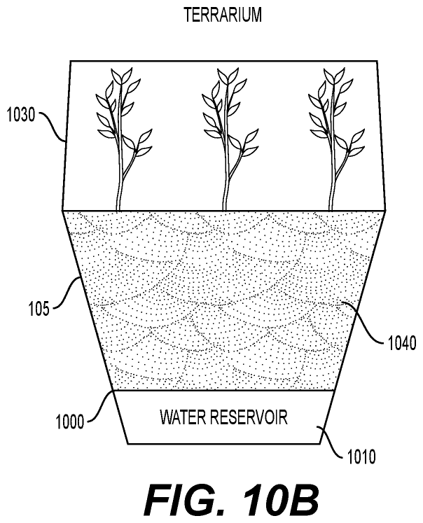

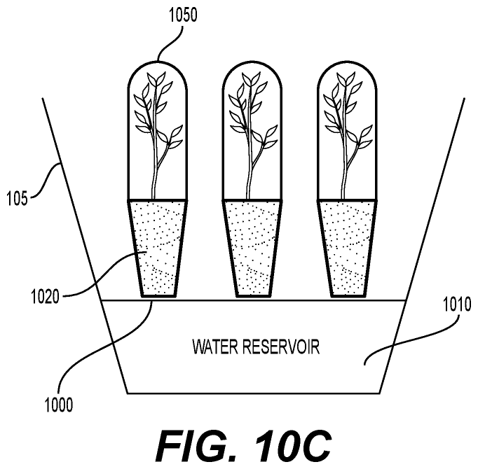

FIG. 10A is an illustration of a greenhouse using a dome. An insert 1000 is placed inside the base container 105 forming a water reservoir 1010. The base container includes a rotatable water spout (not shown) to adjust the level of water in the reservoir 1010. Individual grower pots 1020 are place on the insert 1000, and a dome 1030 is placed on the base container 105 covering the individual grower pots 1020. FIG. 10B is an illustration of a single grower pot 1040 placed on the insert 1000 in the base container 105, and having a dome 1030. FIG. 10C is an illustration of each of the individual grower pots 1010 having its own dome 1050.

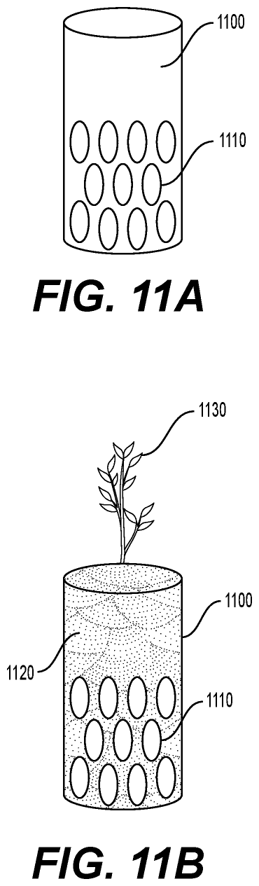

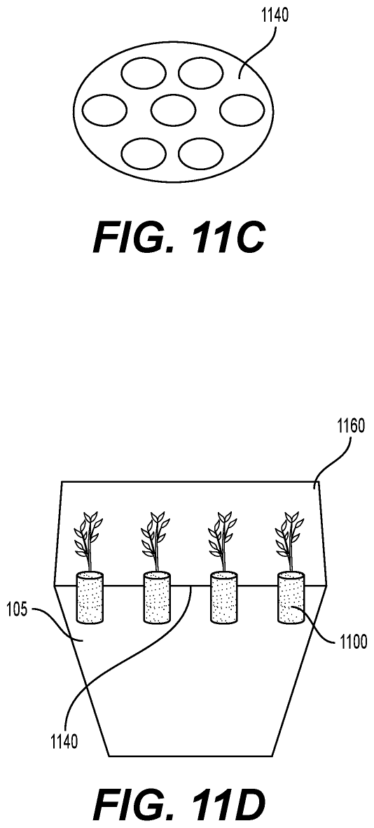

FIG. 11A is an illustration of an individual grower container 1100 having holes 1110 in around the bottom of the container. FIG. 11B is an illustration of the grower container 1100 filled with soil 1120 and a plant 1130. FIG. 11C is an illustration of an insert 1140 with openings 1150 for holding multiple containers 1100. FIG. 11D is an illustration of the insert 1140 with multiple grower containers 1100 placed inside the base container 105 and a dome 1160 covering the base container 105.

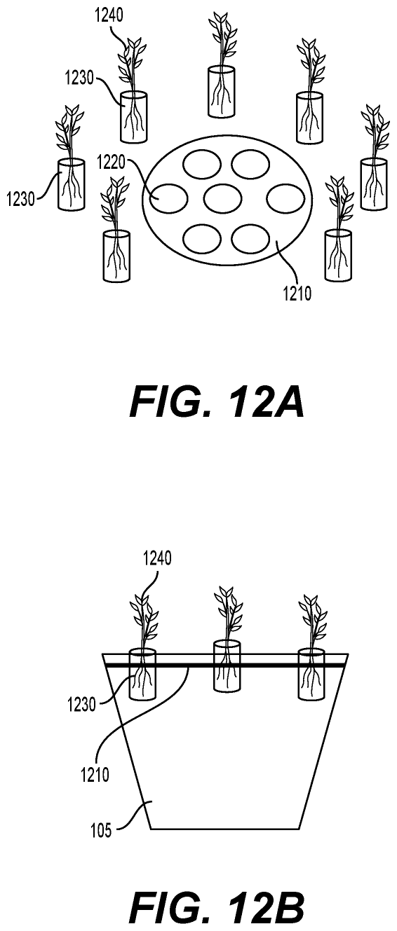

FIG. 12A is an illustration of a slotted insert 1210 with multiple openings 1220 that hold open-ended breakaway containers 1230. The open-ended breakaway containers 1230 hold immature plants 1240 until they are mature and ready for transplanting. FIG. 12B is an illustration of the slotted insert 1210 placed inside the base container 105 which is filled with water. The slotted insert 1210 can include interconnections along the perimeter to interlock with the outer container handles 220 or the base container handles 225. Alternately, the slotted insert 1210 can include pegs along the perimeter, which fit into holes in the outer container handles 220 or the base container handles 225.

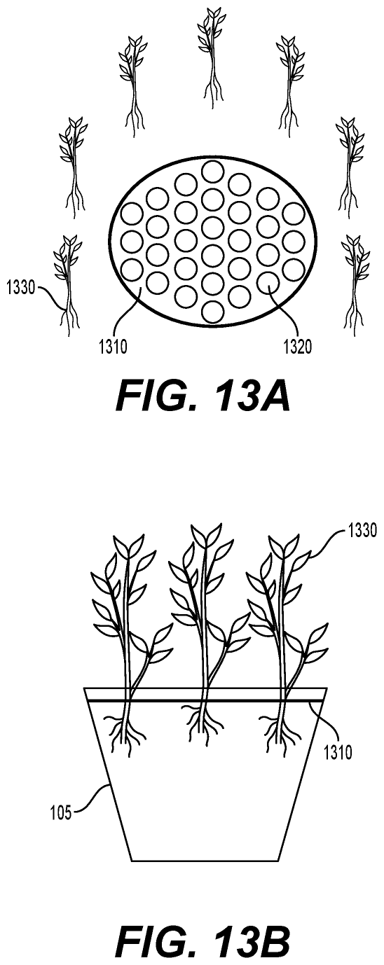

FIG. 13A is an illustration of a slotted insert 1310, with multiple openings 1320 that hold immature plants 1330 without individual containers. FIG. 13A is an illustration of the slotted insert 1310 placed inside the base container 105 which is filled with water.

The base containers 105, as described, can be used with the outer container 125. The outer container 125 and the outer container gate 205 can be joined to form a completely enclosed perimeter as illustrated in FIG. 2C. However, the area where the container and gate are joined leaves an opening 210 for the transparent rotating water evacuation tube 110 to protrude through the outer container 125.

Because the transparent rotating water evacuation tube 110 is connected to the base container 105 through a hole located near the bottom of the transparent base container 105, water can be removed via the opening in the tube from the container by gravity by simply rotating the tube. When the tube is upright, the water level in the base container 105 can be viewed in the transparent rotating water evacuation tube 110.

The feeding/watering/aeration tube 120 is attached to the platform/transplanter insert 115 through a hole in the bottom of the platform/transplanter insert 115.

The wicking tubes 135 that create the raised area of the platform should be packed with soil. The packed soil enhances the wicking effect by creating a conduit for the water to flow from the reservoir into the plant root area. The platform/transplanter insert 115 should be covered with a thin layer of soil. Then, the plant and soil media are placed on the platform/transplanter insert 115.

The base container 105 is inserted into outer container 125 by aligning the transparent rotating water evacuation tube 110 with the opening 210 in the side of outer container 125.

The platform/transplanter insert 115 is placed into the base container 105. Soil is then tightly packed around the platform legs 130. The platform/transplanter insert 115 is covered with soil. A plant is then placed in the base container 105 on the platform/transplanter insert 115. The remaining space is filled with soil which is moistened by adding water. The plant is watered and fed while monitoring the water level through the transparent rotating water evacuation tube 110. The transparent base container 105 allows users to visually inspect and monitor root development and the overall health of the plant. Users can accurately and precisely know exactly how much water to add by monitoring the water level through the transparent rotating water evacuation tube 110. The plant can then be irrigated and fed when necessary.

When watering the plant, users have the option of watering through the soil by pouring water onto the soil at the top of the base container 105 and letting water trickle down through the soil into the reservoir or pouring water directly into the reservoir via the feeding/watering/aeration tube 120 and allowing the water to wick up through the one or more wicking tubes 135. Feeding is easier and more efficient, because plant food can be dropped directly into the reservoir where it is dissolved into the water before it is extracted through the one or more wicking tubes 135. If overwatering accidentally occurs or after intentional soil saturation, water can be removed from the reservoir via the transparent rotating water evacuation tube 110 by simply rotating and allowing gravity to remove the excess water. This feature also prevents surface damage or messes caused by spillovers. From a conservation standpoint, water can be extracted from the reservoir via the transparent rotating water evacuation tube 110, and used to water another plant or re-water the host plant. A user can, also, easily observe when the water reserves have been depleted by visually monitoring the transparent rotating water evacuation tube 110. Better aeration takes place because both the transparent rotating water evacuation tube 110 and the feeding/watering/aeration tube 120 allow fresh, circulating air into the root area of the plant. The air mixes with the water before the water is absorbed into the plant via the one or more wicking tubes 135. Users now know when and how much to water the plant because watering is monitored in real time. Additionally, overwatering mistakes are easily corrected and there are no spills. The planter system, as described, prevents water damage to surfaces caused by spillovers.

When transplanting, a user simply grasps the handles 118 of the platform/transplanter insert 115 and lifts and removes the plant and platform from the base container 105 and places it on a surface outside of the base container 105. This process is less traumatic for the plant, easier for the user, more efficient, and less damaging to the container. The platform/transplanter insert 115 and platform legs 130 will support the plant in an upright position for inspection and maintenance before transplanting the plant into a new container.

From an aesthetic standpoint, the outer container 125 of the planter system can be made of any material and have any design or color scheme. The containers are interchangeable which allows a plant to be presented in a new or different container easily and without any trauma. The plant and base container 105 can be transferred to a different type, color, style, etc. of outer container 125 to change the appearance, if desired. The potential design, material, and color schemes and/or themes are truly unlimited. Users can also forgo the outer container 125 altogether and visually enjoy the natural beauty of the plant, soil, and developing plant root system in a transparent or opaque base container 105.

Currently, in typical container designs, the poured then stored water evaporates into the atmosphere without the plant absorbing the water. In addition, most users will not water their plants when there is water stored in the saucer which often causes the soil to become too dry, while waiting for the water to evaporate from the saucer. In the planter system, as described, the plants extract water from the reservoir via the one or more wicking tube 135. Additionally, less water is lost through evaporation because there is less exposed surface area from which water can evaporate.

A user, currently, cannot know when a container plant has become root bound because there is no way of knowing how well the root system has developed or if it is overgrown. The planter system, as described, allows user to remove the outer container and visually inspect the root system and overall health of the plant while the plant remains in the base container 105.

Transplanting plants using current systems is very traumatic for container plants and often shocks, stuns, and sometimes kills the plant during the process. The new planter system, as described herein, allows the plant to be gently removed from its current container without tugging on the plant. It is then moved to its new location without trauma or difficulty. When transplanting, a user simply grasps the handles 118 and lifts, then removes the plant from the base container 105. This process is less traumatic for the plant, easier for the user, and less damaging to the container.

From aesthetic and efficiency standpoints, container plants are routinely and traditionally placed in bland container/saucer pairings that are neither visually appealing nor efficient from a survival standpoint. The planter system, as described, allows for unlimited designs and materials to be used as presentation materials, and allows the host container's appearance to be easily changed. The outer container 125 of the planter system can be made of any material and have any design or color scheme. The potential design, material, and color schemes and/or themes are truly unlimited. The containers are also interchangeable which allows a plant to be presented in a new or different container easily and without any trauma.

Below are issues associated with typical, conventional container plant care that are addressed and improved upon.

Current systems have inefficient water usage, because water often flows through the soil into or over the container saucers, and is never used by the plant, because of a high evaporation percentage or spillovers. Water is now stored in an enclosed reservoir, that lessens evaporation and eliminates spillovers.

The planter system, as disclosed, allows users to know when and how much water to use, because watering is monitored in real time. Overwatering damages or kills plants. The planter system, as disclosed, allows overwatering mistakes to be easily corrected, and there are no spills. Improper irrigation is eliminated.

Feeding is easier and more efficient because plant food can be dropped directly into the reservoir via the feeding/watering/aeration tube 120, where it is dissolved into the water, and is extracted by the plant through the one or more wicking tube 135.

A user can monitor root development through visual inspections of the root system; can easily check the overall health of plant; and know if and when the plant becomes root bound.

The planter system, as disclosed, allows plants to be saturated with water and then easily removes excess water.

A user can forgo the outer container 125 and use only a transparent base container 105 to visually enjoy the plant root system in addition to the plant itself. The base container 105 can be decorated/personalized as desired.

The planter system, as disclosed, prevents water damage to container host surfaces caused by overwatering spillovers. It also encourages strong root systems, because the plants seek water stored in the reservoir, instead of the roots lingering near the upper edge of plant containers. Better aeration takes place, because both the transparent rotating water evacuation tube 110 and the feeding/watering/aeration tube 120, allow fresh circulating air into the root area of the plant. The air mixes with the water before the water is absorbed into the plant via the one or more wicking tube 135.

It is to be understood that the planter system is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

I claim:

1. A planter system comprising,
a base container;
a water evacuation tube located at a bottom portion of the base container, the water evacuation tube movable from a position pointing up holding water in the base container and a position pointing down to drain water from the base container; and
a platform/transplanter insert located in the base container.

2. The planter system as recited in claim 1, further comprising handles that extend upward.

3. The planter system as recited in claim 1, wherein the handles extend upward along the base container providing a passage for watering, aeration and feeding.

4. The planter system as recited in claim 1, wherein the base container is transparent.

5. The planter system as recited in claim 1, further comprising:
an outer container having a slot that accommodates the transparent water evacuation tube when the base container is located in the outer container; and
an outer container gate that slides into the slot down to the transparent water evacuation tube to form a completely enclosed outer container perimeter.

6. The planter system as recited in claim 1, wherein the platform/transplanter insert has legs extending downward from the bottom portion of the insert leaving space between the bottom portion of the insert and the base container.

7. The planter system as recited in claim 1, wherein the platform/transplanter insert comprises one or more wicking tubes extending into the base container creating a conduit for water to flow from the base container up through the platform/transplanter insert.

8. The planter system as recited in claim 1, wherein the platform/transplanter insert further comprises handles for lifting the platform/transplanter insert.

9. The planter system as recited in claim 1, wherein the platform/transplanter insert comprises two or more vermiculture containers each having matching holes allowing worms to move back and forth.

10. The planter system as recited in claim 1, wherein the platform/transplanter insert comprises two or more mini containers each having holes on a bottom portion allowing water to enter and drain.

11. The planter system as recited in claim 1, further comprising a dome covering the platform/transplanter insert to control humidity and aid in plant germination.

12. A planter system comprising,
a base container;
a water evacuation tube located at a bottom portion of the base container, the water evacuation tube movable from a position pointing up holding water in the base container and a position pointing down to drain water from the base container; and
a platform insert located in the base container providing a space between the bottom of the base container and the platform insert.

13. The planter system as recited in claim 12, wherein a grower cage is attached to the platform insert.

14. The planter system as recited in claim 12, wherein the planter insert further comprises multiple slots.

15. The planter system as recited in claim 14, further comprising one or more grower pots located in one or more of the multiple slots.

16. The planter system as recited in claim 14, further comprising one or more grower containers located in one or more of the multiple slots.

17. The planter system as recited in claim 14, further comprising one or more breakaway containers located in one or more of the multiple slots.

18. The planter system as recited in claim 12, further comprising a dome covering the platform insert to control humidity and aid in plant germination.
